SEQUENCE LISTING (3 sequences)

SEQ ID NO: 1 - protein, 37 aa, Artificial Sequence [sequence omitted]

SEQ ID NO: 2 - protein, 37 aa, Artificial Sequence
  misc_feature (3) .. (6): Description of Artificial Sequence Amylin-Mimetic. Xaa may be an amino acid substitution
KCXXXXCATQRLANFLVHSSNNFGAILSSTNVGSNTY

SEQ ID NO: 3 - protein, 37 aa, Artificial Sequence
  Description of Artificial Sequence Amylin-Mimetic
KCNTATCATQRLANFLVHSSNNFGPILPPTNVGSNTY

(19) United States
(12) Patent Application Publication
(10) Pub. No.: US 2016/0184401 A1
(43) Pub. Date: Jun. 30, 2016

(54) NON-AGGLOMERATING BIOCONJUGATES OF AMYLIN AND AMYLIN-MIMETIC COMPOUNDS, COMPOSITIONS COMPRISING THE SAME, AND MAKING AND USE THEREOF

(71) Applicant: UNIVERSIDADE FEDERAL DO RIO DE JANEIRO, Rio de Janeiro (BR)

(72) Inventors: Luis Mauricio TRAMBAIOLI DA ROCHA E LIMA, Rio de Janeiro (BR); Luiz Henrique GUERREIRO ROSADO, Seropedica (BR); Mariana Fernandes de AVILA NETTO GUTERRES, Rio de Janeiro (BR); Bruno MELO VIEIRA GONÇALVES FERREIRA, Rio de Janeiro (BR)

(21) Appl. No.: 14/968,033

(22) Filed: Dec. 14, 2015

Related U.S. Application Data
(63) Continuation-in-part of application No. PCT/BR2014/000199, filed on Jun. 13, 2014.

(30) Foreign Application Priority Data
Jun. 14, 2013 (BR) 10 2013 017626-5

(51) Int. Cl. A61K 38/22; A61K 45/06; A61K 38/28; C07K 14/575; A61K 47/48

(52) U.S. Cl. CPC A61K 38/22; C07K 14/575; A61K 47/48215; A61K 38/28; A61K 45/06

(57) ABSTRACT

The present disclosure concerns new non-agglomerating bioconjugates of amylin, amylin-mimetic compounds, and combinations comprising the same. Methodology of making and using are provided herein. In some embodiments, an instant non-agglomerating amylin bioconjugate can be used for treating a disease associated with a lack of natural production of amylin and/or deposition or accumulation of extracellular amyloid fibers, which contribute to the dysfunction or failure of systemic organs such as the pancreas or the brain.

### CROSS-REFERENCE TO RELATED APPLICATIONS

This application is a continuation in part of PCT/BR2014/000199, filed Jun. 13, 2014, which application claims priority benefit of BR 10 2013 017626-5, filed Jun. 14, 2013, each application incorporated by reference herein in its entirety.

### FIELD

The present description generally concerns new non-agglomerating bioconjugates of amylin, amylin-mimetic compounds, and combinations comprising the same. Methodology of making and using are provided herein.

### INTRODUCTION

Amylin, also known as Islet Amyloid Polypeptide, identified with CAS RN: 106602-62-4, is a 37-amino acid polypeptide hormone produced by the beta-cells of the pancreatic Langerhans islets. Under physiological conditions, amylin and insulin are produced and stored in the same secretory granules at relatively low pH environment. Both hormones are co-secreted in a proportion insulin:amylin of 15:1 in response to feeding, and amylin plays important physiological roles in glucose homeostasis that are independent of the insulin pathway.

The beneficial activities of amylin in diabetes and obesity are known: increase in satiety leading to reduced ingestion of food and consequent body weight reduction; slower gastric emptying, improved glucose metabolism profiles with postprandial peaks and reduction of glucagon levels in diabetic patients (See Hay et al. Pharmacological reviews, v. 67, n. 3, p. 564-600, 2015).

Type 1 diabetes patients have practically no natural amylin production, while patients with long-standing type 2 diabetes have lower levels than healthy individuals. Since amylin response to meal intake is absent or severely impaired in diabetic patients, some combinations of amylinomimetics and insulin or other anti-diabetic drugs are candidates to restore patient's physiology, which is the main goal of diabetes treatment. Moreover, the glucose control in diabetic patients with the current insulin-based medications is usually suboptimal and invariably induces important side effects (e.g., weight gain and high risk of hypoglycemia).

Amylin replacement treatment in this context may make it possible for induction of satiety, slowed gastric emptying or fewer postprandial glycemia peaks, leading to weight loss and improved control of glucose levels in the long run. However, the use of structurally unmodified amylin as a pharmaceutical ingredient is not possible due to its intrinsic physicochemical properties (i.e., poor solubility, tendency to form amyloid fibers and low plasma stability). Its limited solubility in aqueous solution led to the development of a more soluble amylin-mimetic compound, pramlintide (Symlin®), in which amino acids 25, 27, and 28 were replaced by prolines.

Although the solubility of pramlintide is greater than that of human amylin, pramlintide does not have good stability in neutral pH, so the product Symlin® is provided as an acid solution. This product has to be administered as subcutaneous injections right before the meals, aiming to increase postprandial levels of amylin. Because of its short plasma half-life, from 10 to 15 min, such injections of pramlintide increase the concentration of amylin in the blood stream as a series of peaks, and therefore it did not recover patient's physiology. In the scientific literature, pramlintide has been associated with increased risk of severe insulin-induced hypoglycemia, and other adverse effects such as nausea, vomiting, anorexia and fatigue.

### SUMMARY

In one aspect, provided herein is a non-agglomerating bioconjugate of human amylin or amylin analogue, wherein said bioconjugate contains at least one acyl unit. In one embodiment, the one acyl unit is covalently bonded to the Lysine 1 residue of SEQ ID NO: 1.

In another embodiment, the non-agglomerating bioconjugate of human amylin is represented by formula I

(R1-X)m-R2

wherein R1 represents an acyl moiety selected from the group consisting of acetyl, biotin, ubiquitin, glycans, fatty acid chains and natural or synthetic polymers, polyethylene glycol (PEG), and functional spacers with various average molar masses,

R2 represents SEQ ID NO: 1,

X represents NH, CO, or O,

m represents the number of units of the acyl groups (R1) conjugated to SEQ ID NO: 1 (R2) obtained from the acylation of SEQ ID NO: 1, including human amylin analogues bearing an amino acid substitution in one or more of positions 3, 4, 5 or 6 (SEQ ID NO: 2);

In a further embodiment, the acyl group is produced with at least one acylating agent is selected from the group consisting of PEG an acetyl group, palmitoyl, and myristoyl. In a still further embodiment, PEG is from 1 KDa to 40 Kda.

In another aspect, the disclosure provides a composition comprising a non-agglomerating acylated bioconjugate of human amylin or amylin analogue. In one embodiment, the composition further comprises insulin, an insulin analogue, and/or a GLP-1 receptor agonist analogue; and a pharmaceutically acceptable carrier. In a further embodiment, the insulin or insulin analogue is selected from the group consisting of NPH, regular insulin, Glargine, Detemir, Degludec, Aspart, Lispro, and Glulisine. In another further embodiment, the GLP-1 receptor agonist analogue is selected from the group consisting of semaglutide, exenatide, lixisenatide, liraglutide, albiglutide, and dulaglutide. In another embodiment, the composition further comprises an anti-inflammatory.

In one embodiment, the composition comprising a non-agglomerating acylated bioconjugate of human amylin or amylin analogue is used in the treatment of pancreatitis, hypercalcemia, pain, osteoporosis, chronic inflammatory diseases, coeliac disease, psoriasis, diseases or conditions caused or favored by amyloid deposition or accumulation that leads to dysfunction or failure of systemic organs, and disorders and vascular diseases resulting from increase in blood pressure.

In a further embodiment, the diseases or problems caused or favored by amyloid deposition or accumulation that leads to dysfunction or failure of systemic organs are selected from the group consisting of hyperglycemia, diabetes, low tolerance to glucose or deficient glucose metabolism, obesity, metabolic syndrome, central nervous system-associated or peripherally-associated feeding disorders and Alzheimer's disease.

In a further embodiment, the problems and vascular diseases resulting from increase in blood pressure are selected from the group consisting of atherosclerosis, myocardial infarction, stroke, coronary heart disease, hypertension, and cardiac diseases.

In another embodiment, the present disclosure provides methodology for preparing a composition comprising a non-agglomerating acylated bioconjugate of human amylin or amylin analogue, comprising mixing the non-agglomerating bioconjugate of human amylin, insulin, and a pharmaceutically acceptable carrier.

In another embodiment, there is provided a medicament comprising a therapeutically effective amount of the non-agglomerating bioconjugate of human amylin, wherein one acyl unit is covalently bonded to the Lysine 1 residue of SEQ ID NO: 1.

In another embodiment, provided herein is methodology for treating a disease or condition caused by a lack of amylin or amyloid deposition or accumulation, comprising administering to a patient in need thereof the composition, further comprising insulin, an insulin analogue, and/or a GLP-1 receptor agonist analogue; and a pharmaceutically acceptable carrier. In one embodiment, the disease is diabetes and/or obesity.

In another aspect, provided is methodology for stabilizing an amylin-mimetic compound, comprising binding an acylating agent at the alpha and/or epsilon amine moieties of the lysine 1 residue of the amylin polypeptide chain.

### BRIEF DESCRIPTION OF DRAWINGS

FIG. 1 is a chromatogram for monopegylated amylin obtained by the reaction of human amylin and mPEG 5000 Da (hAmyPEG5k).

FIG. 2 is a mass spectrometry showing characterization of hAmyPEG5k.

FIG. 3 is a chromatogram for monopegylated amylin obtained by the reaction of human amylin and mPEG 20000 Da (hAmyPEG20k).

FIG. 4 is a mass spectrometry showing characterization of hAmyPEG20k.

FIG. 5 and FIG. 6 are assays for RAMP2 and RAMP3, respectively, coreceptor binding interaction for free and purified monopegylated human amylin.

FIG. 7 is an assay for amyloid aggregates.

FIG. 8A-C are plasma stability comparison between free amylin (8A—hAmy) and monopegylated human amylins (8B—hAmyPEG5k; 8C—hAmyPEG20k).

FIG. 9A is an isoelectric focusing of acetylated derivative of human amylin (AcO-hAmy).

FIG. 9B is an isoelectric focusing of hAmyPEG5k.

FIG. 10A show the short-term effect of pH on the aggregation rate of human amylin.

FIG. 10B show the short-term effect of pH on the aggregation rate of hAmyPEG5k.

FIG. 11A shows the short-term effect of pH on the aggregation rate of human amylin.

FIG. 11B shows the short-term effect of pH on the aggregation rate of AcO-hAmy.

FIG. 12 shows the long-term effect of pH on the aggregation rate of hAmyPEG5k.

FIG. 13A-D show the short-term stability of human amylin or its monoPEGylated derivative with recombinant insulins: kinetics of aggregation for human amylin and its PEGylated derivative (A), the selected insulin analogues (B), combinations of insulin analogues with human amylin (C) and hAmyPEG5k (D).

FIG. 14A-B show the long-term stability of the combination of hAmyPEG5k and insulin analogues measured by Dynamic Light Scattering (DLS) for 6 months.

FIG. 15A-B show the long-term stability of the combination of hAmyPEG5k and insulin analogues measured by Thioflavin T binding (Tht) for 6 months.

FIG. 16A-C show the long-term stability of the combination of hAmyPEG5k and insulin Glargine measured by HPLC for 6 months.

FIG. 17A-C show the long-term stability of the combination of hAmyPEG5k and insulin Detemir measured by HPLC for 6 months.

### DETAILED DESCRIPTION

The use of structurally unmodified amylin as a pharmaceutical ingredient is not possible due to its inherent physiochemical properties, such as poor solubility, low plasma stability, and tendency to form amyloid fibers. As explained below, the present inventors developed methodology, compositions, and the like that allow amylin to act as a pharmaceutical ingredient, as well as conjugate amylin with other compounds, such as insulin.

The present inventors made the surprising discovery that conjugating an acylating agent to human amylin improved amylin peptide stability in solutions with neutral pH, and therefore allows amylin bioconjugates to mix with other compounds, such as insulins. At the same time, they realized that the acylating agent may be chosen in order to modulate the pharmacokinetic features of the amylin peptide.

As explained below, the inventors determined that modulating the isoelectric point (pI) of human amylin consequently changes the pH zone in which the amylin peptide aggregates, thereby permitting amylin to combine with other compounds, such as insulin. Until now, mixing amylin or amylin analogues with other compounds in aqueous solutions was impossible due to pI incompatibility. For example, most insulin-based products are formulated near physiological pH, around pH 7.2, while human amylin and its analogues are completely unstable at such pH range. Even insulin glargine, which has pH 4.0, could not successfully combine with amylin analogues (i.e. Pramlintide) due to inappropriate molecular interactions. See, for example, U.S. Application Publication No. 2009-0018053.

Accordingly, the present disclosure contemplates conjugating human amylin or amylin analogues and an acylating agent, a method of making and the use of obtained bioconjugates towards the restoration of the physiologic levels of amylin or the inhibition of extracellular formation of amyloid aggregates, without inducing the toxicity related to the formation of amylin oligomers and clusters.

In one embodiment, the present disclosure provides conjugation of human amylin or a defined range of amylin analogues with an acylating agent.

In another embodiment, the present disclosure provides combinations of bioconjugates of amylin with insulin or other compounds such as but not limited to antidiabetic compounds and/or anti-inflammatory compounds, methods of preparing the same, and uses thereof. In some embodiments, the anti-inflammatory compounds are chosen from steroids and non-steroidal anti-inflammatories (NSAIDS). In some embodiments, the steroids are chosen from prednisone, dexamethasone, and hydrocortisone. In some embodiments, the steroids are corticosteroids chosen from prednisolone, prednisone, medrol, beclomethsone, budesonide, flunisolide, fluticasone and triamcinolone. In some embodiments, the anti-inflammatory compounds are corticosteroids chosen from dexamethasone, mometasone, and triamcinolone. In some embodiments, the NSAIDS are chosen from celecoxib, diclofenac, diflunisal, etodolac, fenoprofen, flurbirofen, ibuprofen, indomethacin, ketroprofen, ketorolac, mefenamic acid, meloxicam, nabumetone, naproxen, oxaprozin, piroxicam, sulindac, and tolmetin.

As used herein, amylin refers to the Islet Amyloid Polypeptide, identified with CAS RN: 106602-62-4. Amylin is a 37-amino acid polypeptide hormone produced by the beta-cells of the pancreatic Langerhans islets. Unless stated otherwise, amylin means human amylin, including natural or synthetic amylin. Amylin encompasses polypeptides of SEQ ID NO: 1 or SEQ ID NO: 2, produced by natural, synthetic or bio-semi-synthetic means.

Amylin-mimetic compounds or amylinomimetics refer to drugs that act or mimic the function of naturally occurring amylin. Amylin and amylin-mimetics are used interchangeably with human amylin, produced naturally, synthetically, or bio-semi-synthetically. Amylin-mimetic encompasses active derivatives of said amylin-mimetic compounds such as salts, isomers, hydrates, solvates, prodrugs, metabolites, polymorphs, and isosteres. It is important to remember that mention of amylin-mimetic compounds encompasses polypeptides of SEQ ID NO: 1 or SEQ ID NO: 2, produced by natural, synthetic, or bio-semi-synthetic means. Pramlintide is a commercially available amylin-mimetic compound, having the sequence set forth in SEQ ID NO: 3.

Acylation refers to the process of quenching a positive charge of amine groups by the insertion of any acyl agent. An acylating agent or acyl agent includes organic uncharged radicals, such as acetyl, biotin, ubiquitin, glycans, fatty acid chains, and natural or synthetic polymers. Polyethylene glycol (PEG) is an exemplary acyl agent.

Non-aglommerating as used herein means that the instant amylin and amylin-mimetic compounds do not agglomerate, polymerize, or otherwise cluster. A non-aglommerating amylin refers to an instant amylin having at least one acyl unit, covalently bound.

Bioconjugation is a chemical strategy to form a stable covalent link between two molecules, at least one of which is a biomolecule. A bioconjugate is the resultant product formed from the two molecules. Throughout this disclosure, several terms refer to formation of a bioconjugate, including mixing, binding, combining, coupling, linking, conjugating, and the like. These terms are used interchangeably and each means bioconjugation.

Analog or Analogue refers to compounds that have similar physical, chemical, biochemical, and/or pharmacological properties. The analog may differ in one or more atoms, functional groups, or substructures, which are replaced with other atoms, groups, or substructures. An example of an analog is insulin and one of NPH, Glargine, Detemir, Aspart, Lispro, Glulisine or Degludec.

Insulin encompasses any type of insulin, including but not limited to natural, synthetic, bio-semi-synthetic or, recombinant insulins. Non limiting examples of insulins include regular insulin, NPH, Glargine, Detemir, Aspart, Lispro, Glulisine or Degludec.

As used herein, a disease or condition caused by lack of amylin or amyloid deposition or accumulation that leads to dysfunction or failure of systemic organs, includes but is not limited to hyperglycemia, diabetes, low tolerance to glucose or deficient glucose metabolism, obesity, metabolic syndrome, central nervous system-associated or peripherally-associated feeding disorders, and Alzheimer's disease.

Scientific and technical terminologies have their usual and ordinary meaning in the art. Pharmaceutical preparations and the like can be found in, for example, “Remington's Pharmaceutical Sciences,” 15^{th} Edition, Mack Publishing Co., New Jersey (1991). Specific formulations, excipients, etc., are chosen according to the desired administration route, within the practice of the pharmacological area.

A. Modification of Amylin

As explained above, the present inventors developed methodology, compositions, and the like that allow amylin or an amylin-mimetic to act as a pharmaceutical ingredient, as well as combine or mix amylin with other compounds, such as an acylating agent and/or insulin.

The present inventors made the surprising discovery that conjugating an acylating agent to human amylin improved amylin peptide stability in solutions with neutral pH, and therefore allows amylin bioconjugates to mix with other compounds, such as insulin. At the same time, they realized that the acylating agent may be chosen in order to modulate the pharmacokinetic features of the amylin peptide.

In this connection, the modification of amylin pI was achieved upon binding an acylating agent to the alpha amine moiety of the lysine 1 residue of the amylin polypeptide chain. According to Bjellqvist (1993), the pI of human amylin can be easily devised from its primary sequence, which results in a pI of 8.9. See Bjellqvist, et al., (1993). The focusing positions of polypeptides in immobilized pH gradients can be predicted from their amino acid sequences. Electrophoresis, 14(1), 1023-1031.

Here, the present inventors determined that coupling an acylating agent with human amylin in the said alpha amine from lys-1, the pI shifts upwards, to a value close to 9.2. The pI of acylated human amylin could range from about 8.9 to about 9.2, and could have a value of about 9.0, 9.1, 9.2, 9.3, 9.4, 9.5, 9.6, 9.7, 9.8, and 9.9.

Accordingly, in one embodiment, the present disclosure contemplates conjugation of human amylin or amylin analogues and an acylating agent, methodology for making such amylin bioconjugates, and the use of said obtained bioconjugates. In one embodiment, an illustrative amylin bioconjugate finds use in the restoration of the physiologic levels of amylin or the inhibition of extracellular formation of amyloid aggregates, without inducing the toxicity related to the formation of amylin oligomers and clusters.

B. Non-agglomerating Amylin: Conjugating Amylin with an Acylating Agent

In one embodiment, the present disclosure contemplates conjugating human amylin or a defined range of amylin analogues with an acylating agent, thereby producing a non-agglomerating amylin.

In a first aspect, the present disclosure provides novel, non-agglomerating bioconjugates of human amylin, with sequence KCNTATCATQRLANFLVHSSNNFGAILSSTNVGSNTY (SEQ ID NO: 1), or amylin-mimetic compounds, with sequence KCX_{AA1}X_{AA2}X_{AA3}X_{AA4}CATQRLANFLVHSSNNFGAILSSTNVGSNTY (SEQ ID NO: 2), and an acylating agent. Pramlintide is another amylin-mimetic compound, commercially available, having the sequence KCNTATCATQRLANFLVHSSNNFGPILPPTNVGSNTY—(NH₂) (SEQ ID NO: 3).

Said bioconjugates may contain, for instance, at least one polyethylene glycol unit (therefore monoconjugated or polyconjugated compounds may be obtained), covalently bound to the nitrogen atom(s) present in the alpha or epsilon amine moieties (lateral chain) of the lysine 1 residue of the amylin polypeptide chain. Furthermore, a person skilled in the art could also replace amino acids X_{AA1}, X_{AA2}, X_{AA3} and/or X_{AA4} with other uncharged or positively charged amino acids and achieve acylated amylin analogues with higher pI than those bearing Lys1 acylation alone.

C. Stabilizing Amylin-Mimetic Compounds

The present disclosure provides methodology for stabilizing amylin-mimetic compounds comprising binding an acylating agent at the alpha and/or epsilon amine moieties (lateral chain) of the lysine 1 residue of the amylin polypeptide chain.

In one embodiment, the non-agglomerating bioconjugates of amylin-mimetic compounds are represented by formula I

(R1-COX)m-R2
where
R1 represents an acylating agent,
R2 represents amylin (SEQ ID NO: 1) or amylin-mimetic (SEQ ID NO: 2) compounds,
X represents NH, CO, or O,
m represents the number of units of the acylating agent (R1) conjugated to amylin-mimetic compounds (R2) obtained from the conjugation of an acylating agent with amylin-mimetic compounds through an amide or ester bond by reaction of the primary amine or the hydroxyl functional moieties. Amylin-mimetics include analogues wherein amino acids in position 3, 4, 5 or 6 have been substituted by positively charged or uncharged amino acids.;
and compounds of formula II

(R1-X)m-R2
where
R1 represents an acylating agent moiety and functional spacers with various average molar masses,
R2 represents amylin (SEQ ID NO: 1) or amylin-mimetic (SEQ ID NO: 2) compounds,
X represents NH, CO, or O,
m represents the number of units of the acylating agent (R1) conjugated to amylin-mimetic compounds (R2) obtained from the conjugation of the acylating agent with amylin-mimetic compounds.

Of course, it is understood that the instant bioconjugate amylin-mimetic compounds encompass active derivatives of said amylin-mimetic compounds such as salts, isomers, hydrates, solvates, prodrugs, metabolites, polymorphs, and isosteres.

It is important to remember that mention of amylin-mimetic compounds encompasses polypeptides of SEQ ID NO: 1 or SEQ ID NO: 2, either natural, synthetic, or bio-semi-synthetic.

The term “alkyl” as used herein alone or as part of another group refers to a straight or branched chain aliphatic hydrocarbon chain, having from 1 to 35 carbon atoms. Examples of alkyl include, but are not limited to methyl, ethyl, n-propyl, isopropyl, n-butyl, n-pentyl, t-butyl and the like. Alkyl groups may further be substituted with one or more suitable substituents.

The term “alkenyl” as used herein alone or as part of another group refers to a straight or branched chain aliphatic hydrocarbon group containing at least one carbon-carbon double bond, having from 2 to 35 carbon atoms. Examples of alkenyl include, but are not limited to ethenyl, 1-propenyl, 2-propenyl, iso-propenyl, 1-butenyl, 2-butenyl, and the like. Alkenyl groups may further be substituted with one or more suitable substituents.

The term “alkynyl” as used herein alone or as part of another group refers to a straight or branched chain aliphatic hydrocarbon group containing at least one carbon-carbon triple bond, having from 2 to 35 carbon atoms. Examples of alkynyl include, but are not limited to ethynyl, propynyl, and butynyl. Alkynyl groups may further be substituted with one or more suitable substituents.

The term “cycloalkyl” refers to cyclic alkyl groups constituting of 3 to 20 carbon atoms having a single cyclic ring or multiple condensed rings, for example, fused or spiro systems, unless otherwise constrained by the definition. Such cycloalkyl groups include, by way of example, single ring structures, for example, cyclopropyl, cyclobutyl, cyclopentyl, cyclohexyl, cyclooctyl, and the like, or multiple ring structures, for example, adamantyl, and bicyclo[2.2.1]heptane, or cyclic alkyl groups to which is fused an aryl group, heteroaryl group, heterocyclyl group or another cycloalkyl group, for example, indane and the like. Cycloalkyl groups may further be substituted with one or more suitable substituents.

The term “cycloalkenyl” refers to a cycloalkyl group as defined above which may optionally contain one or more double bonds.

The term “cycloalkynyl” refers to a cycloalkyl group as defined above which may optionally contain one or more triple bonds.

The term “heterocyclyl” unless otherwise specified refers to a non-aromatic monocyclic or polycyclic cycloalkyl group, fully or partially unsaturated, constituting of 5 to 15 carbon atoms, with one or more heteroatom(s) independently selected from N, O, S or P. The nitrogen, sulphur and phosphorus heteroatoms may optionally be oxidized. The nitrogen atoms may optionally be quaternerized. The heterocyclyl group may be further substituted at any available position with one or more suitable substituents. Examples of heterocyclyl groups include but are not limited to, morpholinyl, oxazolidinyl, tetrahydrofuranyl, dihydrofuranyl, dihydropyridinyl, dihydroisooxazolyl, dihydrobenzofuryl, azabicyclohexyl, dihydroindonyl, piperidinyl or piperazinyl.

The term “aryl” herein refers to a mono- or poly-carbocyclic aromatic group constituting of 5 to 15 carbon atoms, for example phenyl or naphthyl ring and the like optionally substituted with one or more suitable substituents. The aryl group may optionally be fused with cycloalkyl group, heteroaryl group, heterocyclyl group or another aryl group. The fused group may be further substituted with one or more suitable substituents.

The term “heteroaryl” unless and otherwise specified refers to an aromatic monocyclic or polycyclic ring structure constituting of 5 to 15 carbon atoms, containing one or more heteroatoms independently selected from N, O, S or P. The nitrogen, sulphur and phosphorus heteroatoms may optionally be oxidized. The nitrogen atoms may optionally be quaternerized. “Heteroaryl” also includes, but is not limited to, bicyclic or tricyclic rings, wherein the heteroaryl ring is fused to one or two rings independently selected from an aryl ring, a cycloalkyl ring, a cycloalkenyl ring, a heterocyclyl ring and another monocyclic heteroaryl ring. Examples of heteroaryl groups include, but are not limited to, oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, thienyl, isoxazolyl, triazinyl, furanyl, benzofuranyl, indolyl, benzothiazolyl, benzoxazolyl, imidazo[1,2-a]pyrimidine, imidazo[1,2-a]pyrazine, tetrahydroquinoline and the like. The heteroaryl group may be further substituted at any available position with one or more suitable substituents.

The term “PEG” unless otherwise specified refers to a polyethylene glycol and as used herein is meant any water soluble poly(ethylene oxide). The term PEG includes the structure—(CH₂CH₂O)ₙ— where n is an integer ranging from 2 to 1000 or from 2 to 500 or from 2 to 250 or from 2 to 125 or from 2 to 50 or from 2 to 25 or from 2 to 12. A commonly used PEG is end-capped PEG, wherein one end of the PEG termini is end-capped with a relatively inactive group such as alkoxy, while the other end is a hydroxyl group that may be further modified by linker moieties. An often used capping group is methoxy and the corresponding end-capped PEG is often denoted mPEG. Hence, mPEG is CH₃O(CH₂CH₂O)ₙ—, where n is an integer from 2 to about 1000 sufficient to give the average molecular weight indicated for the whole PEG moiety, e.g., for mPEG Mw 2,000, n is approximately 44±10.

Specific PEG forms are branched, linear, forked, dumbbell PEGs, and the like and the PEG groups are, in some embodiments, polydisperse, possessing a low polydispersity index of less than about 1.05. The PEG moieties for a given molecular weight typically consist of a range of ethyleneglycol (or ethyleneoxide) monomers. For example, a PEG moiety of molecular weight 2000 will typically consist of 44±10 monomers, the average being around 44 monomers. The average molecular weight (and number of monomers) will typically be subject to some batch-to-batch variation.

Other specific PEG forms are monodisperse that can be branched, linear, forked, or dumbbell shaped as well. Being monodisperse means that the length (or molecular weight) of the PEG polymer is specifically defined and is not a mixture of various lengths (or molecular weights).

The term alkoxy covers “alkyl-O—” wherein alkyl is as defined above. Representative examples are methoxy, ethoxy, propoxy (e.g., 1-propoxy and 2-propoxy), butoxy (e.g., 1-butoxy, 2-butoxy and 2-methyl-2-propoxy), pentoxy (1-pentoxy and 2-pentoxy), hexoxy (1-hexoxy and 3-hexoxy), and the like.

D. Illustrative Amylin-Mimetic Compounds

Illustrative amylin-mimetic compound are chosen from those having SEQ ID 1, SEQ ID 2, or SEQ ID 3, in which the alpha and/or epsilon amino acid of lysine-1 has a hydrogen atom replaced with an acyl moiety Q chosen from

—C(═Y)—R¹; and

—C(═Y)—R²—PEG,
in which:
Y is O or S; and
R¹, R², and PEG are defined herein.

In some embodiments, Y is O. In some embodiments, Y is S.

In some embodiments, the amylin-mimetic compound are chosen from those having SEQ ID 1, SEQ ID 2, or SEQ ID 3, in which either the alpha or the epsilon amino acid of lysine-1 (but not both) has a hydrogen atom replaced with an acyl moiety Q. In some embodiments, the amylin-mimetic compound are chosen from those having SEQ ID 1, SEQ ID 2, or SEQ ID 3, in which the both the alpha and the epsilon amino acid of lysine-1 have a hydrogen atom replaced with an acyl moiety Q.

Types of —C(═Y)—R¹

In some embodiments, Q is a an acyl moiety —C(═Y)—R¹;

in which:

R¹ represents C₁₋₃₅ alkyl, C₂₋₃₅ alkenyl, C₂₋₃₅ alkynyl, C₃₋₂₀ cycloalkyl, C₃₋₂₀ cycloalkenyl, C₆₋₂₀ cycloalkynyl, heterocyclyl, aryl, heteroaryl; each of which may be optionally substituted at any available position by from one or more substituents independently selected from C₁₋₁₂ alkyl, C₂₋₁₂ alkenyl, C₂₋₁₂ alkynyl, C₃₋₇ cycloalkyl, C₃₋₇ cycloalkenyl, C₆₋₈ cycloalkynyl, heterocyclyl, aryl, heteroaryl, ═O, —CN, —COCN, —N₃, —NO₂, —OCN, —NCO, —SCN, —NCS, —OCONH₂, —ONO₂, —F, —Cl, —Br, —I, —CHO, —CHS, —COOH, —COSH, —CONH₂, —CONHNH₂, —CSNHNH₂, —CSNH₂, —NH₂, —NHCONH₂, —NHCSNH₂, —N(C═NH)NH₂, —NHNH₂, —NHCHO, —NHCHS, —NHCOOH, —NHCSOH, —OH, —SH, —SO₃H, —CH(═NOH), —CH(═NCN), —COR^{a}, —CSR^{a}, —COOR^{a}, —CSOR^{a}, —COSR^{a}, —CONR^{a}R^{b}, —CSNR^{a}R^{b}, —COCOR^{a}, —CONR^{a}NR^{b}R^{c}, —CSNR^{a}NR^{b}R^{c}, —CSNR^{a}R^{b}, —NR^{a}R^{b}, —NR^{a}SO₂R^{b}, —NR^{a}CONR^{b}R^{c}, —NR^{a}CSNR^{b}R^{c}, —NR^{a} (C═NR^{b})NR^{c}R^{d}, —NR^{a}NR^{b}R^{c}, —NR^{a}COR^{b}, —NR^{a}CSR^{b}, —NR^{a}COOR^{b}, —NR^{a}CSOR^{b}, ═NOR^{a}, —OR^{a}, —OCOR^{a}, —OCOOR^{a}, —OCONR^{a}R^{b}, —OCSR^{a}, —OCSOR^{a}, —ONO₂, —OCSNR^{a}R^{b}, —SR^{a}, —S(O)R^{a}, —S(O)₂R^{a}, —SO₂NR^{a}R^{b}, —CR^{a} (═NOR^{b}), —CR^{a} (═NCOOR^{b}), —CR^{a} (═NSOR^{b}), —CR^{a} (═NSO₂R^{b}), —C(═NR^{a})—NR^{b}R^{c}, —C(═NOR^{a})—NR^{b}R^{c}, —CR^{a} (═NCN), —NCR^{a}, —P(O)R^{a}R^{b}, —P(O)OR^{a}OR^{b}, —P(O)R^{a}OR^{b}, —P(O)NR^{a}OR^{b}, —P(O)NR^{a}R^{b}, —OP(O) R^{a}R^{b}, —NHP(O) R^{a}R^{b};

R^{a}, R^{b}, R^{c} and R^{d} are independently selected from —H, C₁₋₁₂ alkyl, C₂₋₁₂ alkenyl, C₂₋₁₂ alkynyl, C₃₋₇ cycloalkyl, C₃₋₇ cycloalkenyl, C₆₋₈ cycloalkynyl, heterocyclyl, aryl, heteroaryl, ═O, —CN, —COON, —N₃, —NO₂, —OCN, —NCO, —SCN, —NCS, —OCONH₂, —ONO₂, —F, —Cl, —Br, —I, —CHO, —CHS, —COOH, —COSH, —CONH₂, —CONHNH₂, —CSNHNH₂, —CSNH₂, —NH₂, —NHCONH₂, —NHCSNH₂, —N(C═NH)NH₂, —NHNH₂, —NHCHO, —NHCHS, —NHCOOH, —NHCSOH, —OH, —SH, —SO₃H, —CH(═NOH), —CH(═NCN); or
R^{a} and R^{b} are joined together to form a C₃₋₇ cycloalkyl, C₃₋₇ cycloalkenyl, C₆₋₈ cycloalkynyl, heterocyclyl, aryl, heteroaryl; or
R^{b} and R^{c} are joined together to form a C₃₋₇ cycloalkyl, C₃₋₇ cycloalkenyl, C₆₋₈ cycloalkynyl, heterocyclyl, aryl, heteroaryl; or
R^{c} and R^{d} are joined together to form a C₃₋₇ cycloalkyl, C₃₋₇ cycloalkenyl, C₆₋₈ cycloalkynyl, heterocyclyl, aryl, heteroaryl.

In some embodiments, R¹ represents C₁₀₋₃₀ alkyl, C₁₀₋₃₀ alkenyl, C₁₀₋₃₀ alkynyl, C₅₋₁₂ cycloalkyl, C₅₋₁₂ cycloalkenyl, C₈₋₁₆ cycloalkynyl, heterocyclyl, aryl, heteroaryl; each of which may be optionally substituted as noted above.

In some embodiments, R¹ represents C₁₂₋₁₈ alkyl, C₁₂₋₁₈ alkenyl, C₁₂₋₁₈ alkynyl, C₆₋₁₀ cycloalkyl, C₆₋₁₀ cycloalkenyl, C₁₀₋₁₄ cycloalkynyl, heterocyclyl, aryl, heteroaryl; each of which may be optionally substituted as noted above.

In some embodiments, R¹ may be optionally substituted at any available position by from one, two, three four, five, or six substituents independently selected from those noted above.

In some embodiments, Q is chosen from acyl moieties in which R¹ represents C₁₋₆ alkyl and is optionally substituted as noted above. For example, in some embodiments, Q is acetyl and is optionally substituted as noted above.

In some embodiments, Q is chosen from acyl moieties in which R¹ represents C₁₋₃₅ alkyl, C₁₀₋₃₀ alkyl, and C₁₂₋₁₈ alkyl and is optionally substituted as noted above. For example, in some embodiments, Q is chosen from acyl moieties of lauric acid (CH₃(CH₂)₁₀CO—), myristic acid (CH₃(CH₂)₁₂CO—), palmitic acid (CH₃(CH₂)₁₄CO—), stearic acid (CH₃(CH₂)₁₆CO—), and arachidic acid (CH₃(CH₂)₁₈CO—), and the acyl moieties are optionally substituted as noted above.

In some embodiments, Q is chosen from acyl moieties in which R¹ represents C₂₋₃₅ alkenyl, C₁₀₋₃₀ alkenyl, and C₁₂₋₁₈ alkenyl and is optionally substituted as noted above. For example, in some embodiments, Q is chosen from acyl moieties of palmitoleic acid (CH₃(CH2)₅CH═CH(CH₂)₇CO—), oleic acid (CH₃(CH₂)₇CH═CH(CH₂)₇CO—), linoleic acid (CH₃(CH₂)₄CH═CHCH₂CH═CH(CH₂)₇CO—), linolenic acid (CH₃CH₂CH═CHCH₂CH═CHCH₂CH═CH(CH₂)₇CO—), and arachidonic acid (CH₃(CH₂)₄(CH═CHCH₂)₄(CH₂)₂CO—), and the acyl moieties are optionally substituted as noted above.

In some embodiments, Q is chosen from acyl moieties in which R¹ represents C₂₋₃₅ alkynyl, C₁₀₋₃₀ alkynyl, and C₁₂₋₁₈ alkynyl and is optionally substituted as noted above. For example, in some embodiments, Q is chosen from acyl moieties in which R¹ represents ethynyl, propynyl, and butynyl, and the acyl moieties are optionally substituted as noted above.

In some embodiments, Q is chosen from acyl moieties in which R¹ represents C₃₋₂₀ cycloalkyl, C₅₋₁₂ cycloalkyl, and C₆₋₁₀ cycloalkyl, and is optionally substituted as noted above. For example, in some embodiments, Q is chosen from acyl moieties in which R¹ represents single ring structures, for example, cyclopropyl, cyclobutyl, cyclopentyl, cyclohexyl, and cyclooctyl; multiple ring structures, for example, adamantyl and bicyclo[2.2.1]heptane, and the acyl moieties are optionally substituted as noted above.

In some embodiments, Q is chosen from acyl moieties in which R¹ represents C₃₋₂₀ cycloalkenyl, C₅₋₁₂ cycloalkenyl, and C₆₋₁₀ cycloalkenyl, and is optionally substituted as noted above. In some embodiments, for example, the cycloalkyenyl has one, two, or three double bonds, and the acyl moieties are optionally substituted as noted above.

In some embodiments, Q is chosen from acyl moieties in which R¹ represents C₆₋₂₀ cycloalkynyl, C₈₋₁₆ cycloalkynyl, and C₁₀₋₁₄ cycloalkynyl, and is optionally substituted as noted above. In some embodiments, for example, the cycloalkynyl has one, two, or three triple bonds, and the acyl moieties are optionally substituted as noted above.

In some embodiments, Q is chosen from acyl moieties in which R¹ represents heterocyclyl constituting of 5 to 15 carbon atoms or of 8 to 12 carbon atoms, with one, two, three, four, five, or six heteroatoms independently selected from N, O, S or P, in which the nitrogen, sulphur and phosphorus heteroatoms may optionally be oxidized, in which the nitrogen atoms may optionally be quaternerized, and the acyl moieties are optionally substituted as noted above. For example, in some embodiments, Q is chosen from acyl moieties in which R¹ represents a hetercyclyl chosen from morpholinyl, oxazolidinyl, tetrahydrofuranyl, dihydrofuranyl, dihydropyridinyl, dihydroisooxazolyl, dihydrobenzofuryl, azabicyclohexyl, dihydroindonyl, piperidinyl and piperazinyl, and the acyl moieties are optionally substituted as noted above.

In some embodiments, Q is chosen from acyl moieties in which R¹ represents aryl constituting of 5 to 15 carbon atoms or of 6 to 10 carbon atoms, and the acyl moieties are optionally substituted as noted above. For example, in some embodiments, Q is chosen from acyl moieties in which R¹ represents aryl chosen from phenyl and naphthyl, and the acyl moieties are optionally substituted as noted above.

In some embodiments, Q is chosen from acyl moieties in which R¹ represents heteroaryl constituting of 5 to 15 carbon atoms or 8 to 12 carbon atoms, containing one, two, three, four, five, or six heteroatoms independently selected from N, O, S or P, in which the nitrogen, sulphur and phosphorus heteroatoms may optionally be oxidized, in which the nitrogen atoms may optionally be quaternerized, and the acyl moieties are optionally substituted as noted above. For example, in some embodiments, Q is chosen from acyl moieties in which R¹ represents heteroaryl chosen from oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, thienyl, isoxazolyl, triazinyl, furanyl, benzofuranyl, indolyl, benzothiazolyl, benzoxazolyl, imidazo[1,2-a]pyrimidine, imidazo[1,2-a]pyrazine, and tetrahydroquinoline, and the acyl moieties are optionally substituted as noted above.

In some embodiments, R¹ is optionally substituted at any available position by from one or more substituents independently selected from heterocyclyl. In some embodiments, the heterocyclyl is constituted of 5 to 15 carbon atoms or of 8 to 12 carbon atoms, with one, two, three, four, five, or six heteroatoms independently selected from N, O, S or P, in which the nitrogen, sulphur and phosphorus heteroatoms may optionally be oxidized, and in which the nitrogen atoms may optionally be quaternerized. For example, in some embodiments, heterocyclyl is chosen from morpholinyl, oxazolidinyl, tetrahydrofuranyl, dihydrofuranyl, dihydropyridinyl, dihydroisooxazolyl, dihydrobenzofuryl, azabicyclohexyl, dihydroindonyl, piperidinyl and piperazinyl.

In some embodiments, R¹ is optionally substituted at any available position by from one or more substituents independently selected from aryl. In some embodiments, the aryl is constituting of 5 to 15 carbon atoms or of 6 to 10 carbon atoms. For example, in some embodiments, aryl is chosen from phenyl and naphthyl.

In some embodiments, R¹ is optionally substituted at any available position by from one or more substituents independently selected from heteroaryl. In some embodiments, the heteroaryl is constituting of 5 to 15 carbon atoms or 8 to 12 carbon atoms, containing one, two, three, four, five, or six heteroatoms independently selected from N, O, S or P, in which the nitrogen, sulphur and phosphorus heteroatoms may optionally be oxidized, and in which the nitrogen atoms may optionally be quaternerized. For example, in some embodiments, heteroaryl is chosen from oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, thienyl, isoxazolyl, triazinyl, furanyl, benzofuranyl, indolyl, benzothiazolyl, benzoxazolyl, imidazo[1,2-a]pyrimidine, imidazo[1,2-a]pyrazine, and tetrahydroquinoline.

In some embodiments, moiety Q is chosen from acyl moieties in which R¹ represents acetyl, the acyl moiety of biotin (5-[(3aS,4S,6aR)-2-oxohexahydro-1H-thieno[3,4-d]imidazol-4-yl]pentanoic acid),

The amylin-mimetic compound is prepared, in some embodiments, from the daughter amylin (SEQ ID 1, SEQ ID 2, or SEQ ID 3). The Q moieties can either be attached by nucleophilic substitution (acylation) on the alpha-amino group and/or epsilon-amino group on lysine-1, e.g., with OSu-activated esters of the Q moieties, or the Q moieties can be attached by reductive alkylation—on the alpha-amino group and/or epsilon-amino group on lysine-1—using Q-aldehyde reagents and a reducing agent, such as sodium cyanoborohydride.

E. Conjugating Amylin with Insulin or other Hypoglycemic Peptide

While commercially available amylin-mimetic compound pramlintide exists, it does not formulate well with insulin (Weyer et al., 2005). For example, U.S. Pat. No. 6,410,511 discloses attempts to mix pramlintide and different insulins in the same syringe immediately before administration, but notes the short term stability of the formulations. U.S. Published Patent Application No. 2010/0222251 discloses strategies to associate in the same solution long-acting insulin and amylin peptides, particularly, pramlintide. The co-formulations displayed high aggregation profile, reinforcing the technical unfeasibility of mixing insulin and amylin analogues.

Aside from the physicochemical incompatibilities, clinical trials revealed that pramlintide should not be combined with insulins due to impairments in pharmacokinetics of both compounds when administrated together. See Symlin (Pramlintide acetate) New Drug Application. Division of Pharmaceutical Evaluation-II Office of Clinical Pharmacology and Biopharmaceutics. Available in:

http://www.fda.gov/ohrms/dockets/ac/01/briefing/3761b1_05_Pharmacology.pdf

Thus, it is the present inventors who successfully contemplated combinations of bioconjugates of amylin with insulin or other anti-diabetic and/or anti-inflammatory compounds, methods of preparing the same, and uses thereof.

Accordingly, the present disclosure concerns new non-agglomerating combinations of bioconjugates of amylin or amylin-mimetic compounds with human insulin analogues, wherein said bioconjugates contain at least one molecule of an acylating agent, such as (without excluding any other) polyethylene glycol, acetyl, palmitoyl, myristoyl, or other synthetic polymer (therefore monoconjugate or polyconjugate compounds may be obtained), covalently bonded to the lysine 1 residue of the amylin polypeptide chain, and the insulin analogue is, for example, regular insulin, NPH, Glargine, Detemir, Aspart, Lispro, Glulisine or Degludec. Of course, other insulin analogues may be used.

In another embodiment, the new non-agglomerating bioconjugates of amylin or amylin-mimetic compounds are combined to at least one Glucagon-Like Peptide-1 (GLP-1) receptor agonist, such as semaglutide, exenatide, liraglutide, lixisenatide, albiglutide and dulaglutide. Of course, other GLP-1 receptor agonists may be used.

As described below, the present disclosure concerns the use of the instant non-agglomerating bioconjugates of amylin-mimetic compounds and combinations comprising said bioconjugates and insulin analogues for the treatment of type 1 and type 2 diabetes mellitus.

The instant non-agglomerating bioconjugates of amylin-mimetic compounds and combinations comprising said bioconjugates and GLP-1 receptor agonists may be used in the clinical therapy of obesity.

F. Composition, Pharmaceuticals, and Methods of Use

The present bioconjugates of amylin and/or amylin-mimetic compounds can be used as therapeutic or prophylactic approaches in the management of a variety of conditions that directly or indirectly concern the agglomeration/deposition of amyloid fibers. For example, and in no way limiting, pancreatitis, hypercalcemia, pain, osteoporosis, inflammations, coeliac disease, psoriasis, diseases or problems caused or favored by the amyloid deposition or accumulation that leads to dysfunction or failure of systemic organs, such as hyperglycemia, diabetes, low tolerance to glucose or deficient glucose metabolism, obesity, metabolic syndrome, central nervous system-associated or peripherally-associated feeding disorders and Alzheimer's disease, and indirectly to problems and vascular diseases resulting from increase in blood pressure, such as atherosclerosis, myocardial infarction, stroke, coronary heart disease, hypertension, cardiac diseases in general and (see, e.g., Young. Amylin: physiology and pharmacology. Gulf Professional Publishing, 2005.; Zhu et al. Molecular psychiatry, v. 20, n. 2, p. 252-262, 2015.).

In this way, the instant non-agglomerating bioconjugates of amylin-mimetic compounds and combinations comprising the same can be used for the preparation of products, medicaments, compositions and associations, useful in the prevention or treatment of diseases caused or favored by amyloid deposition or accumulation which leads to dysfunction or failure of systemic organs.

For example, and in no way limiting, the instant bioconjugates described herein, particularly, bioconjugates of human amylin, makes it possible to show various benefits, such as the following:
(1) Increased plasma half-life, compared to amylin and amylinomimetic substances such as pramlintide, therefore allowing longer permanence in the blood stream;
(2) Lower doses or fewer injections to mimic the effect of natural amylin;
(3) Good control of basal and postprandial glycemia; and
(4) Higher solubility than human amylin.

In this way, the instant bioconjugates of human amylin aim to avoid the typical toxicity caused by human amylin and amylin-mimetic compounds, by decreasing or avoiding agglomeration (also mentioned in the literature as polymerization), deposition and fibrillation upon the pancreatic beta-cells and, in consequence, avoiding harmful effects that apoptosis or destruction of said pancreatic beta-cells cause to the human organism.

The present disclosure provides pharmaceutical compositions comprising a therapeutically effective amount of one or more of the instant non-agglomerating bioconjugates of amylin-mimetic compounds and one or more pharmaceutically acceptable excipients. Such compositions are adequate for all variety of administration forms such as but not limited to oral, enteral, parenteral, lingual, sublingual, nasal, dermal, epidermal, transdermal, mucosal, vaginal, rectal, ocular, etc.

Yet in another aspect, the present disclosure contemplates low toxicity pharmaceutical compositions comprising a combination of a therapeutically effective amount of one or more of the instant non-agglomerating bioconjugates of amylin-mimetic compounds and one or more pharmaceutically acceptable excipients and a therapeutically effective amount of one or more insulins. Such compositions are adequate for all variety of administration forms such as but not limited to oral, enteral, parenteral, lingual, sublingual, nasal, dermal, epidermal, transdermal, mucosal, vaginal, rectal, ocular, etc.

Methodology for preparing said compositions are also contemplated. Particularly, the instant compositions present themselves in any necessary or adequate dosage forms, such as solutions, suspensions, emulsions, microemulsions, foams, pastes, creams, tablets, capsules (hard or soft, suppositories), bolus, gels, powders, aerosols, sprays, etc.

The pharmaceutically acceptable excipients employed in the instant compositions are known to the person skilled in the art, such as the ones described, for instance, in “Remington's Pharmaceutical Sciences”, 15th edition, Mack Publishing Co., New Jersey (1991). As known, specific excipients are chosen according to the desired administration route, within the practice of the pharmacological area.

The instant pharmaceutical compositions may additionally comprise one or more active principles, distinct from human amylin, such as but not limited to insulin analogues, ions (such as zinc and sodium), GLP-1 receptor agonists, antidiabetics, antibiotics, anti-inflammatory, anti-hypertensives, antiretrovirals, etc. Such compositions may be of immediate, retarded or slow release, also including the possibility that the administration of the new bioconjugate of human amylin be concomitant or sequential to other active principles.

Still another aspect concerns the use of non-agglomerating bioconjugates of human amylin as an adjuvant in the prevention or treatment of diseases caused or favored by amyloid deposition or accumulation that leads to dysfunction or failure of systemic organs.

Still another aspect concerns a medicament characterized by the fact that it comprises a therapeutically effective amount of one or more bioconjugates of human amylin.

Still another aspect concerns bioconjugates of human amylin, as well as products, medicaments, compositions and associations that comprise them, characterized for the use in medical therapy.

Still another aspect concerns a method of treatment or prevention of diseases caused or favored by amyloid deposition or accumulation, characterized by comprising the administration to a patient of a therapeutically effective amount of one or more bioconjugates of amylin-mimetic compounds, particularly human amylin.

In another aspect, the present disclosure concerns the use of the instant non-agglomerating bioconjugates of amylin-mimetic compounds and combinations comprising the said bioconjugates and insulin analogues for the treatment of type 1 and type 2 diabetes mellitus. The mention of diabetes also encompasses other diseases and dysfunctions directly or indirectly related to the agglomeration/deposition of amyloid fibers

In another aspect, the present disclosure concerns the use of the instant non-agglomerating bioconjugates of amylin-mimetic compounds and combinations comprising the said bioconjugates and GLP-1 receptor agonists in the clinical therapy of obesity.

### EXAMPLES

The following examples concern particular embodiments and do not in any way limit the scope or spirit of the present disclosure. A person of ordinary skill in the art may use the present disclosure and examples to make equivalent embodiments which, though not expressly stated, perform the same or similar functions to attain the same or similar results, and therefore are encompassed by the scope and spirit of the present disclosure.

### Example 1

### Preparation of a Conjugate

Reaction for 2 h at 25° C. of a 5 mg/mL human amylin solution in the presence of 10 mM PBS (phosphate buffer solution) pH 7.4, and a molar excess of 5 mPEG 5000 Da or 20000 Da/1 human amylin.

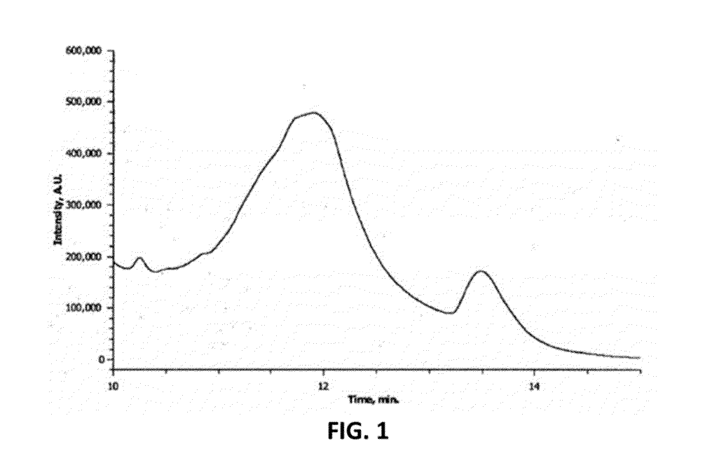

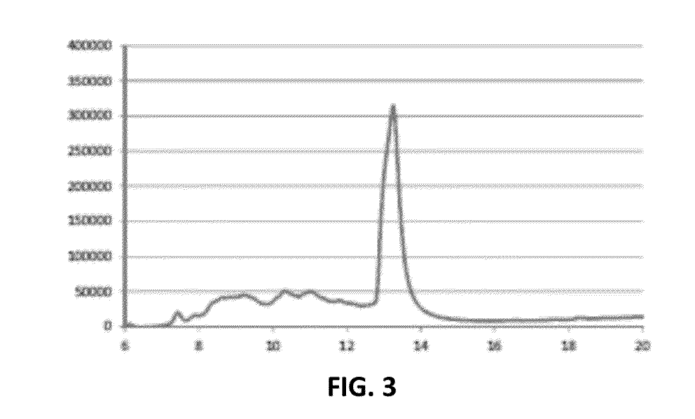

The reaction is quenched by the addition of an equal amount of 30% acetonitrile/0.1% trifluoroacetic acid (in water) and then chromatographed in a C18 Kromasil reversed phase column at 4 mL/min, detector set at 220 nm. (Kromasil is a product line commercialized by Separation Products group, a department of AkzoNobel company, Sweden). The chromatograms of hAmy-PEG5k and hAmy-PEG20k are shown in FIG. 1 and FIG. 3, respectively. The hAmy-PEG5k chromatogram shows a peak of monoPEGylated amylin at 12 minutes. While that for a hAmy-PEG20k is about 13 minutes.

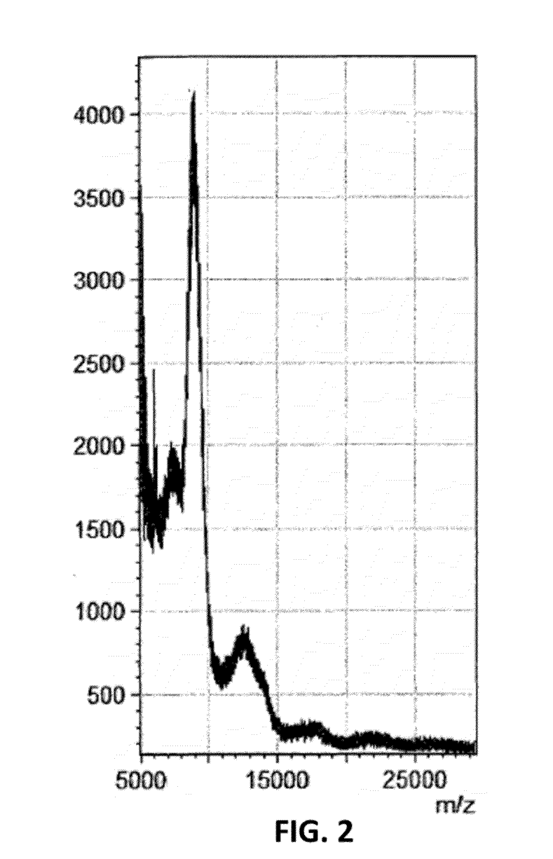

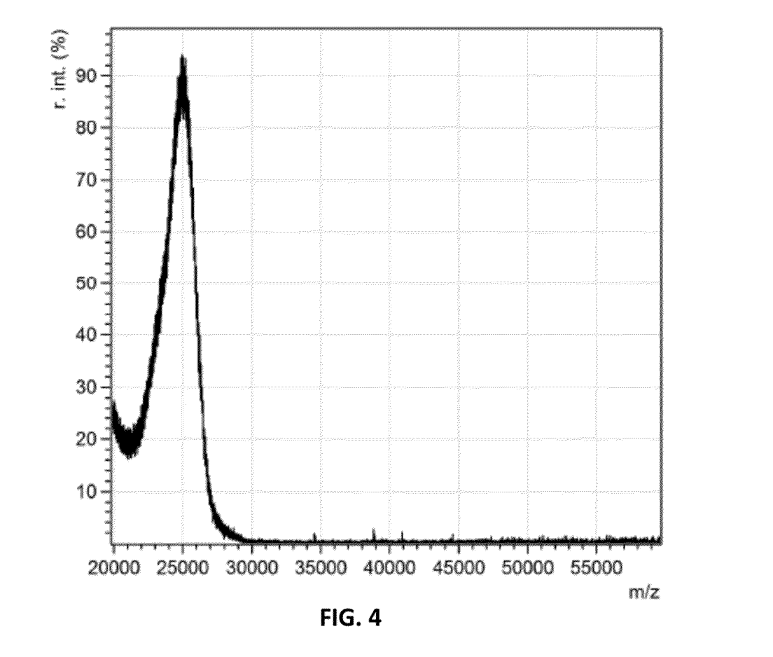

Matrix-assisted laser desorption and ionization-time-of flight mass spectrometry (MALDI-TOF-MS) was performed to characterize monoPEGylated human amylin. FIG. 2 shows the peak of hAmy-PEG5k at 9 KDa and the m/z ratio for hAmy-PEG20k is depicted in FIG. 4.

### Example 2

### Receptor Binding Assay of Human Amylin and MonoPEGylated Human Amylin

Purified monoPEGylated human amylin was assayed for RAMP2 and RAMPS coreceptor binding interaction.

RAMP (receptor activity modifying protein) was labeled with fluorescein isothiocianate for 1 h at 4° C. in PBS (fetal bovine serum) pH 7.4, and purified by size exclusion chromatography (SEC) in Sepharose G25 (agarose-based chromatography media, provided by the US company GE Healthcare) using the same buffer. Labelling was confirmed by UV absorbance measurements at A280 and A490, allowing estimation of coupling efficiency, relying on about 0.5 fluorescein/RAMP molecule. Binding was performed by measuring the fluorescence anisotropy of RAMP-FITC (fluorescein isothiocyanate) as a function of free murine amylin, hAmy-PEG5k and by using the unrelated protein hen egg white lisozyme (HEWL) as a control for non-specific binding.

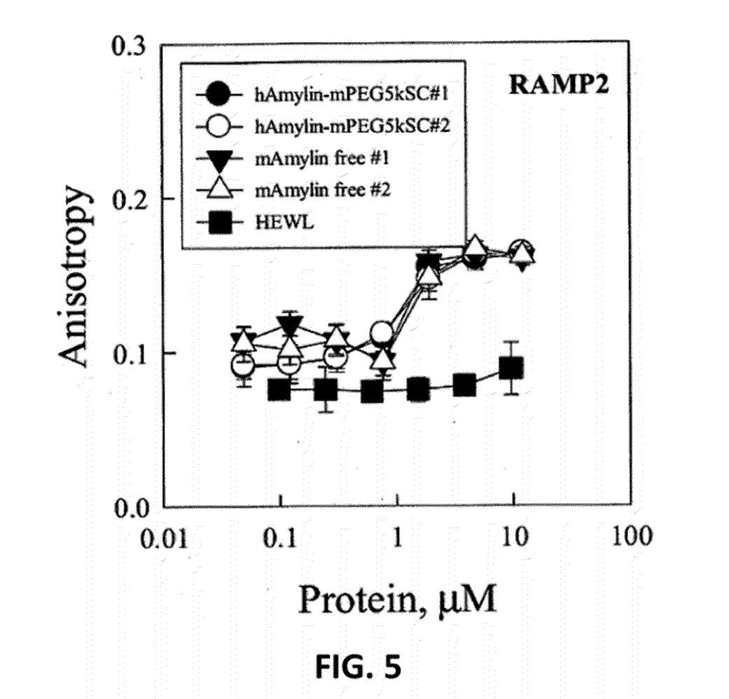

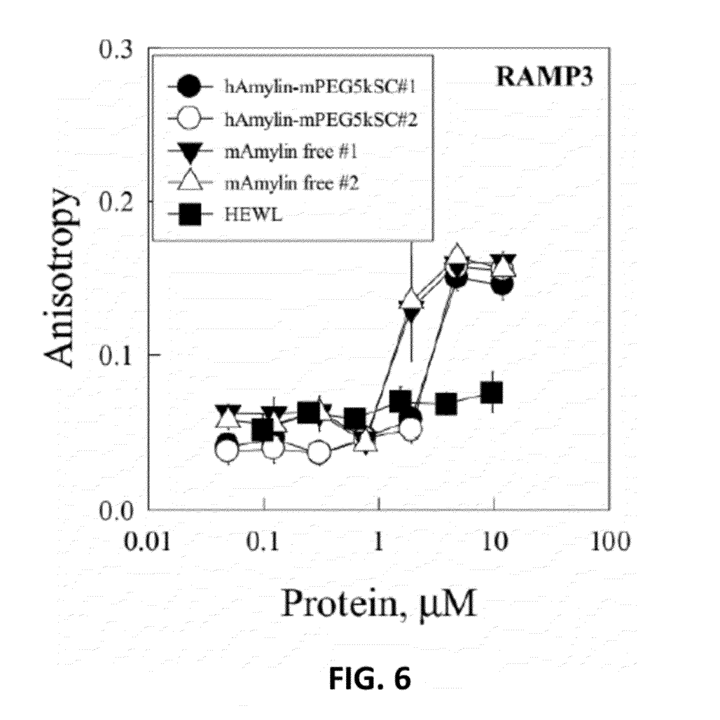

According to FIGS. 5 and 6, binding assay show a similar apparent binding affinity of both free and purified hAmy-PEG5k for the coreceptors RAMP2 and RAMP3, in duplicate assays (monoPEGylated human amylin C#1 and C#2; and free amylin #1 and #2), indicating that the PEG moiety does not interfere with the coreceptor affinity. Control assay using HEWL show nonspecific binding to either coreceptor.

### Example 3

### Effect of PEGylation on the Aggregation Profile of Human Amylin

Purified monoPEGylated human amylin of example 1 was re-suspended in DMSO and diluted to PBS pH 7.4 and allowed to incubate at 37° C. Samples were evaluated for amyloid aggregates by using the amyloid chromogenic probe thioflavin T (ThT). The employment of ThT binding protocols to measure amyloid fibril formation has already been determined in the art (see, e.g., Levine, 1999). The kinetics of amylin (50 μM of free or hAmy-PEG5k) aggregation was performed by incubating the protein in 10 mM NaH₂PO₄ pH 7.4 at 25° C. Aggregation was followed by the binding of Thioflavin T (ThT) to amyloid fibrils, by adding 160 μL sample with 40 μL ThT 100 μM and monitoring fluorescence by exciting at 450 nm and measuring emission at 482 nm for 24 h

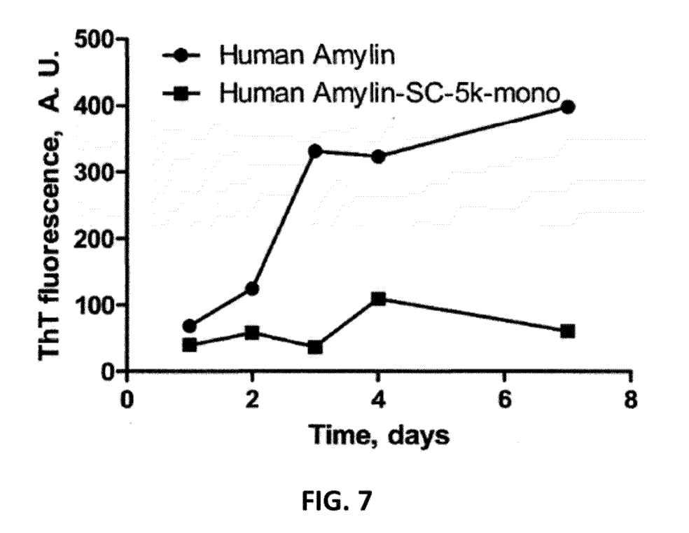

FIG. 7 shows the results. Free human amylin aggregates very fast. In 3 days, almost all free human amylin was aggregated in amyloid form. The hAmy-PEG5k, at day 7, still shows no signs of aggregation. This assay proves that the PEGylation process was able to inhibit the agglomeration of human amylin.

### Example 4

### Pharmacokinetics of the PEGylated Human Amylin Products

The pharmacokinetics of the hAmy-PEG5k and hAmy-PEG20k were characterized in vivo in Harlan Sprague Dawley male rats. The animals were housed in a temperature-controlled room with a light-dark cycle of 12 h. Water and food were available ad libitum. Three groups were formed, control (free human amylin, not PEGylated), hAmy-PEG5k and hAmy-PEG20k. Animals received 500 uL of saline containing 100 μg of human amylin peptides, either free human amylin or purified monoPEGylated human amylin derivatives. Blood was collected retro-orbitally from rats (n=3 rats for each time interval), and the plasma was subjected to ELISA assay for human amylin as provided by the manufacturer (Millipore, Cat Number EZHA-52K.

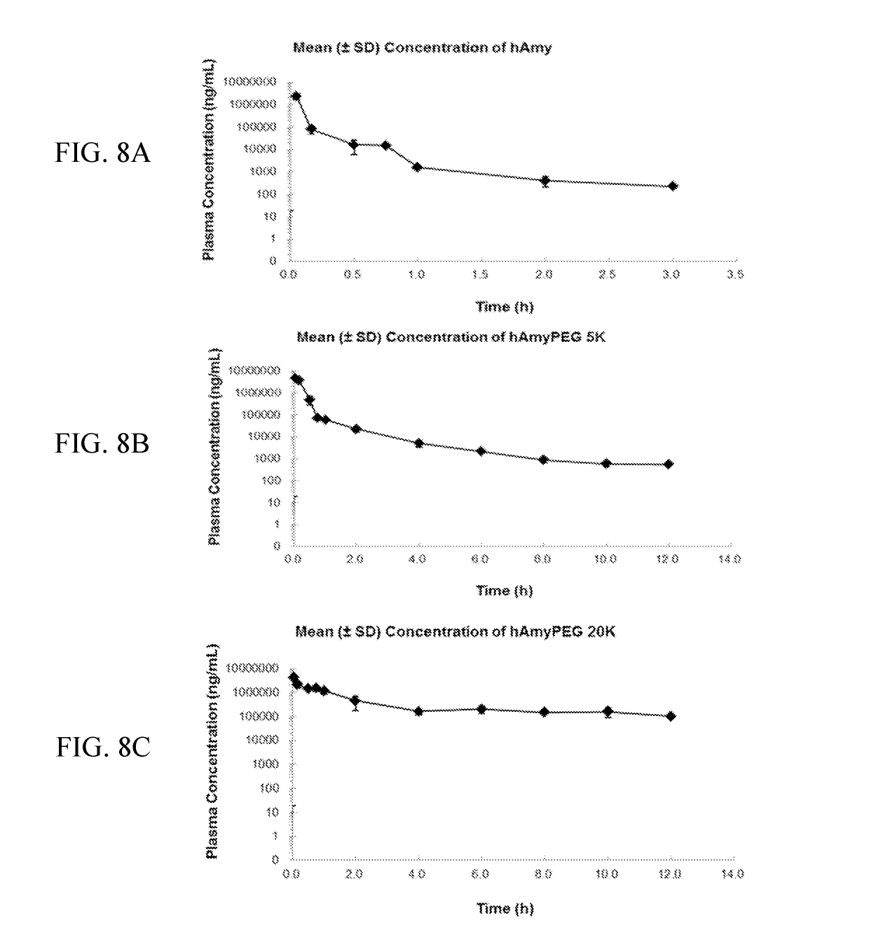

FIG. 8A shows fast plasma decay of free amylin, while the hAmy-PEG5k (FIG. 8B) and hAmy-PEG20k (FIG. 8C) show longer duration of stability in plasma. This assay proves that the process of PEGylation of human amylin is capable of increasing its half-life.

### Example 5

### Effect of Different Acylating Agents on the pI of Human Amylin

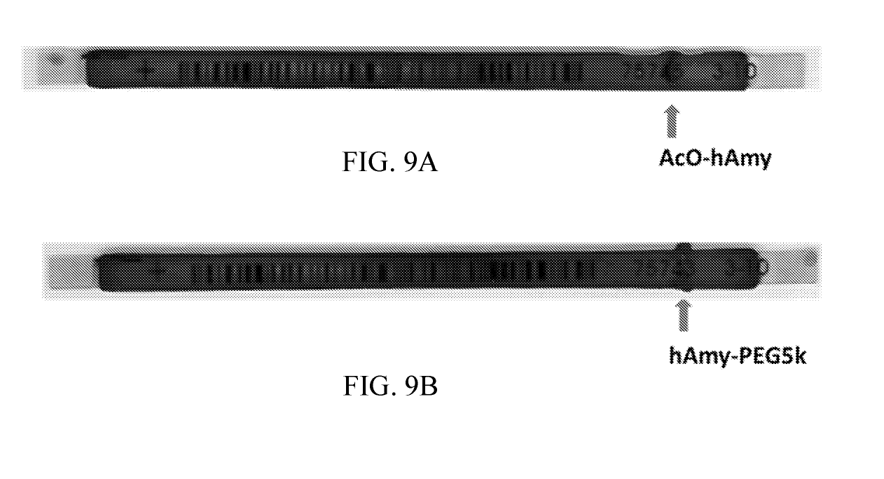

Sixty micrograms of acetylated human amylin (AcO-hAmy) or hAmyPEG5k were dissolved into 8 M urea, 2% CHAPS and 0.002% bromophenol blue buffer to a final volume of 125 μL. The samples were incubated into a 7 cm pH 3-10 linear immobilized pH gradient strips overnight prior to isoelectric focalization (IEF). The IEF was carried out in MultiPhor II (Pharmacia Biotech) device at 20° C. according to the following conditions, 200 V (1 min), ramping to 3500 V (90 min) and 3500 V (60 min). At the end, the strips were fixed with 0.20% glutaraldehyde (in 30% ethanol, 0.2 M sodium acetate) for 60 min at room temperature, and then stained for peptide identification by Coomassie brilliant blue R-250 (at 0.025% in 40% methanol, 7% acetic acid) for 30 min. The excess of Coomassie was removed by incubation with 0.1% acetic acid until no background visualization. The IEF can be easily calculated from the relationship between displacements in the strips and pH. As depicted in FIG. 9, the acetylation of Lys1 present in AcO-hAmy (FIG. 9A) and the PEGylation in the same site, present in hAmy-PEG5k (FIG. 9B), results in similar effects on the pI of human amylin, increasing it to approximately 9.2.

### Example 6

### Effect of Different Acylating Agents on the Aggregation Profile of Human Amylin in Time- and pH-Dependent Settings

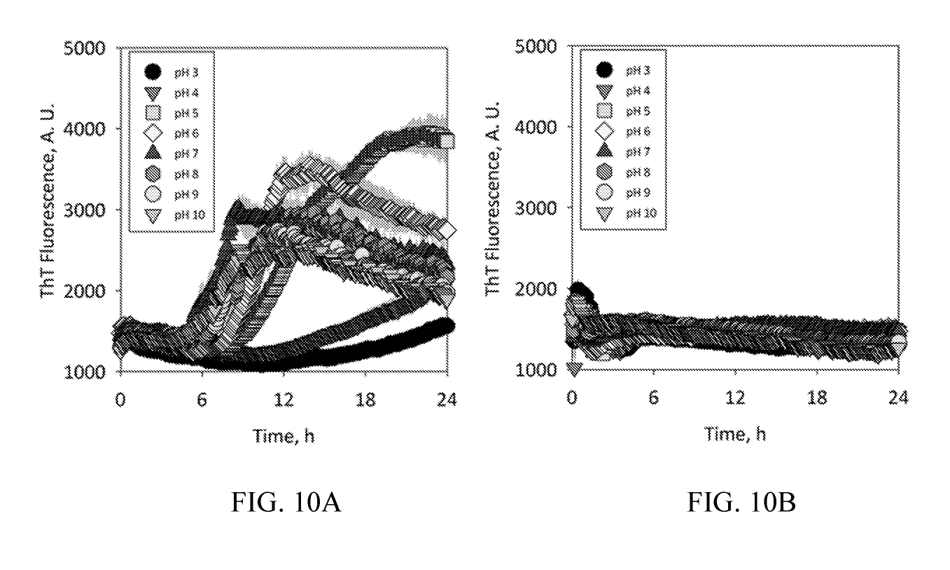

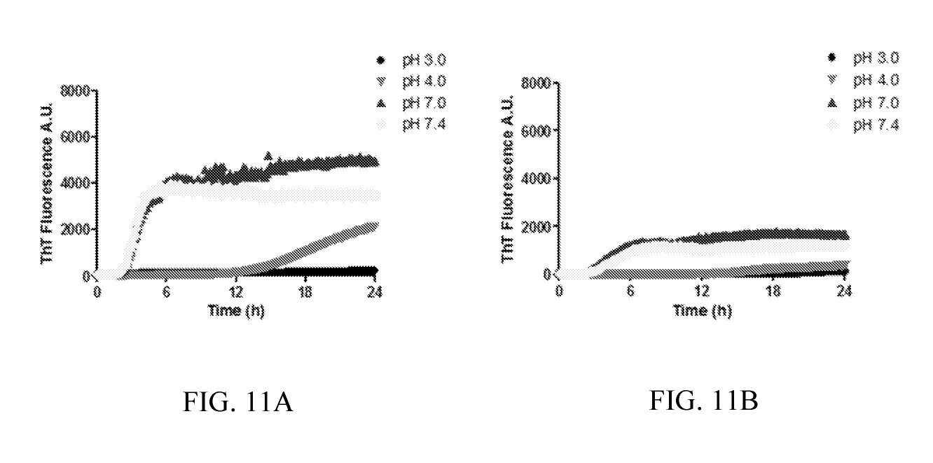

In a first setting, free human amylin, hAmyPEG5k and AcO-hAmy were incubated in aqueous buffered solutions with different pH (from 3.0 to 10.0) in the presence of ThT. Fluorescence emissions derived from ThT binding to amyloid fibers were monitored for 24h at 25° C. The aggregation kinetics of free human amylin were similar to the knowledge available in the art (FIGS. 10A and 11A). However, both the PEGylated and acetylated derivatives of human amylin displayed a pattern of improved aqueous stability and lower or absent formation of amyloid fibers (FIGS. 10B and 11B, respectively). These findings support the hypothesis that acylated derivatives of human amylin have increased stability in aqueous solutions, especially, in physiologic pH ranges.

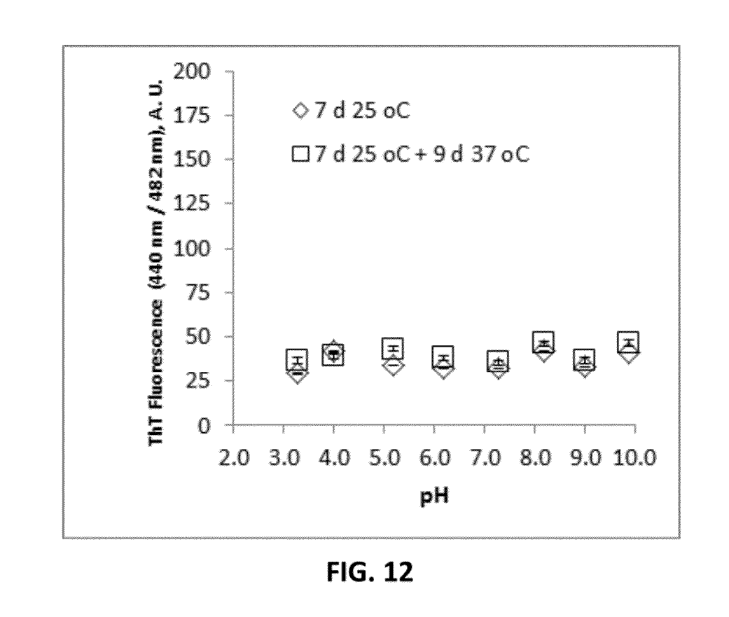

In another experimental setting, the influence of pH over the stability of the hAmyPEG5k was also tested for a longer period of time (up to 16 days) and at higher energy environments (37° C.). FIG. 12 shows the results of the pH effect upon the hAmy-PEG5k after 7 days at 25° C. and thereafter another 9 days at 37° C. (i.e., 16 days total). This assay provides evidence that hAmy-PEG5k remains non-aggregated after several days at different pH, even when subjected to high temperatures.

### Example 7

### Short-Term Stability of hAmy-PEG5K and Insulin Analogues Upon Combination

The aggregation ratio of human amylin or its PEGylated derivative upon combination with insulin R, insulin detemir, or insulin glargine was tested by ThT binding assay. Combinations (10 μM of each compound) were incubated with 10 mM NaH₂PO₄ pH 7.4 and in presence of 10 μM ThT, for 24h at 25° C. Fluorescence variations indicating amyloid fibers formation were monitored by exciting at 450 nm and measuring emission at 482 nm.

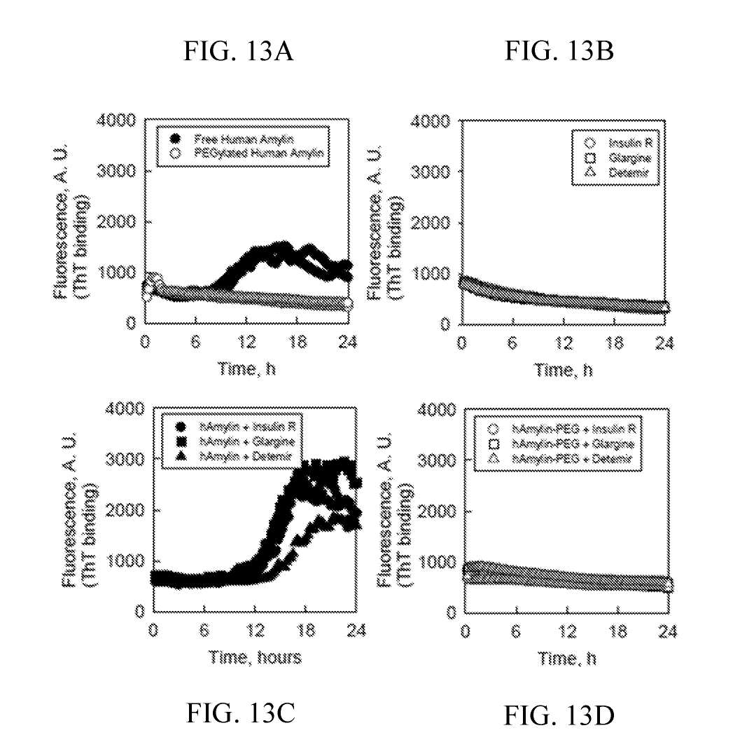

FIG. 13A shows that hAmy aggregates while hAmy5k does not.

FIG. 13B shows insulin R, glargine, and detemir have negligible aggregation.

FIG. 13C shows that human amylin combined with insulin aggregates, but FIG. 13D shows that hAmy-PEG5k combined with insulin does not aggregate.

In addition, this assay proves that the combination of hAmy-PEG5k with insulin remains stable for 24 h.

### Example 8

### Long-Term Stability of MonoPEG-hAmy5k and Insulin Analogues Upon Combination

In these experimental settings, PEGylated human amylin (0.2 mg/mL or 1.0 mg/mL) was added to the original formulation of each selected insulin-based product (glargine and detemir), avoiding major interferences in the insulin regular vehicle. The combinations were manipulated under sterile environment and kept under refrigerated conditions (4° C.) for a 6 month period. Stability of the solutions was measured by ThT-binding assay, dynamic light scattering and transmission electron microscopy. Samples were also analyzed by high performance liquid chromatography to confirm integrity of the active constituents.

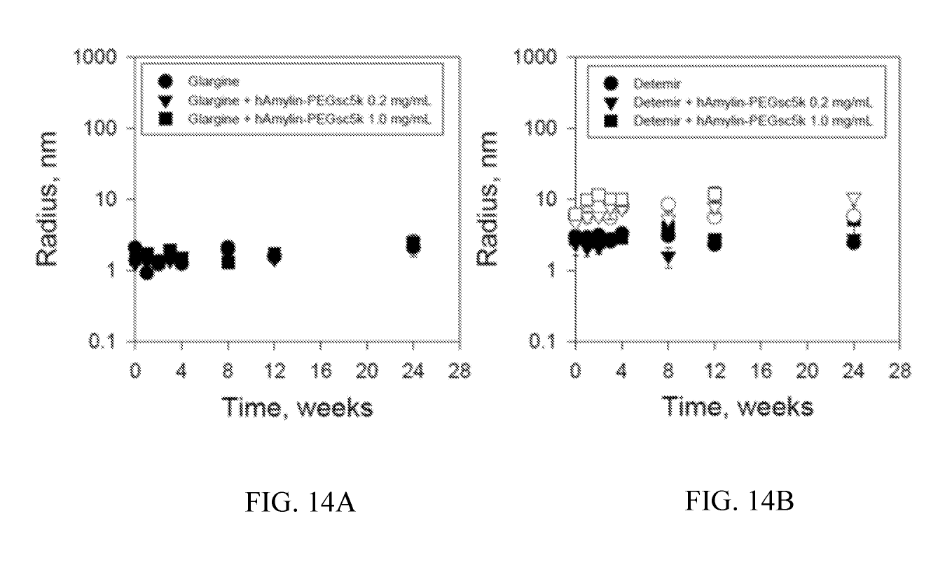

FIG. 14 shows the result of the long-term stability of hAmy-PEG5k and insulin (Glargine and Detemir) by Dynamic Light Scattering. This assay proves that the combination remains stable for 6 months.

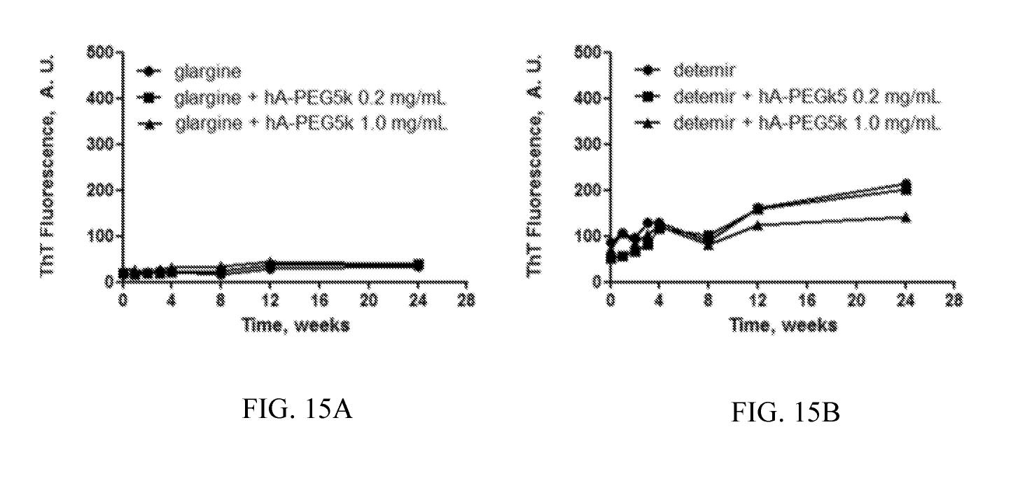

FIG. 15 shows the result of the long-term stability of hAmy-PEG5k and insulin (Glargine and Detemir) by Thioflavin T binding. This assay shows that the combination does not aggregate during 6 months, proving, therefore, that the combination remains stable for 6 months.

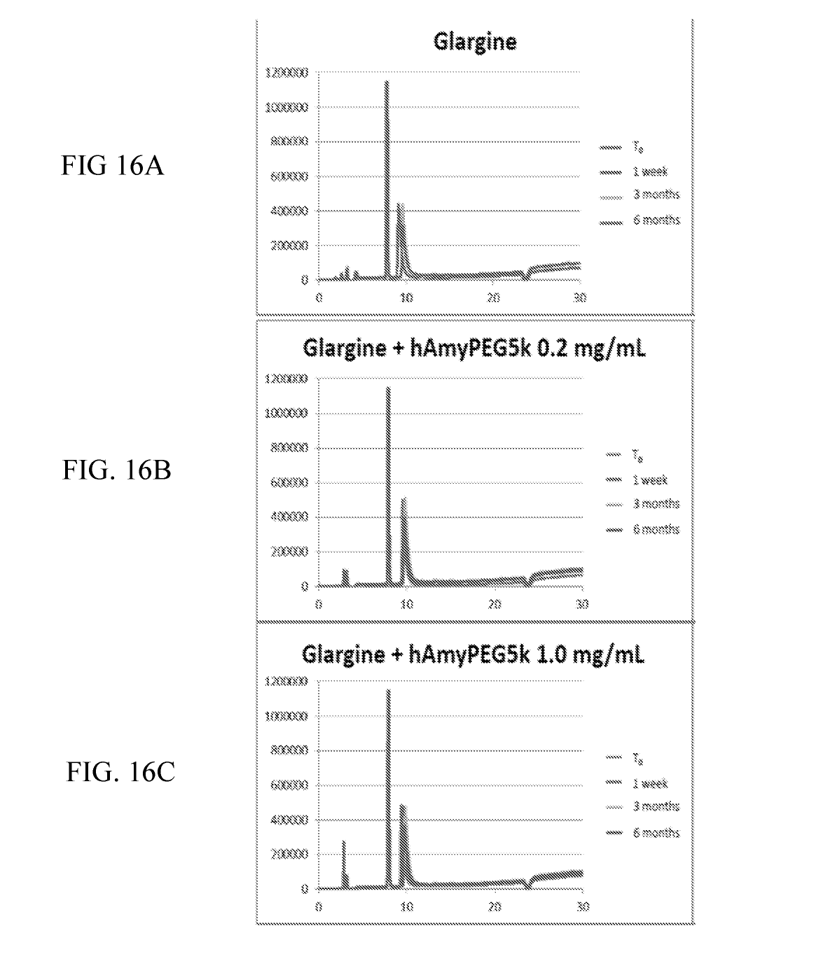

FIG. 16 shows the result of the long-term stability of hAmy-PEG5k and insulin (Glargine) by HPLC. This assay shows that the insulin remains stable for 6 months.

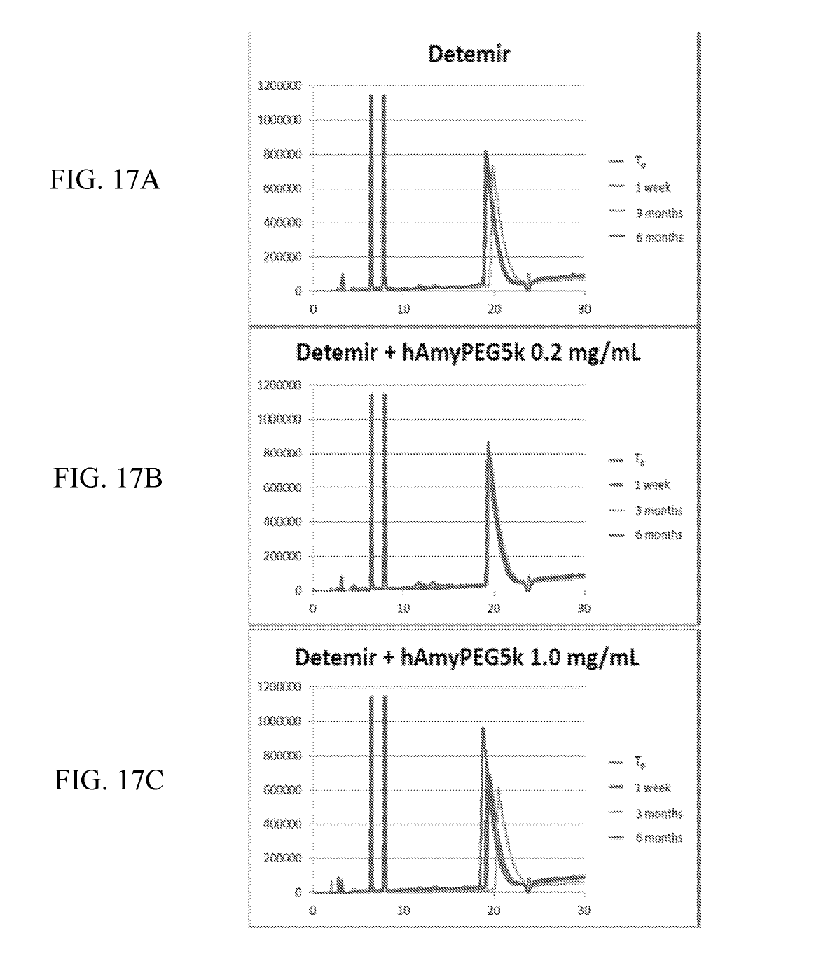

FIG. 17 shows the result of the long-term stability of hAmy-PEG5k and insulin (Detemir) by HPLC. This assay shows that the insulin remains stable for 6 months.

### Example 8.1

### ThT-Binding Assay

The aggregation rate of human amylin or its PEGylated derivative upon combination with insulin levemir or insulin glargine was also tested at 0, 1, 2, 3, 4, 8, 12 and 24 weeks after incubation at 4° C. Combinations were incubated with 10 mM NaH2PO4 pH 7.4 and in presence of 10 μM ThT, for 24 h at 25° C. Fluorescence variations indicating amyloid fibers formation were monitored by exciting at 450 nm and measuring emission at 482 nm.

### Example 8.2

### Dynamic Light Scattering Analysis

Particle size distribution of the human insulin glargine (90 U/mL) and hAmyPEG5k (concentration between 0.2 and 1.0 mg/mL), alone and mixed-up, were evaluated by dynamic light scattering in a DynaPro NanoStar (Wyatt Technology, USA). A 658 nm laser wavelength was used to detect backscattered light at a fixed angle of 90°. The cell holder was maintained at 25° C. throughout the experiment. The data was collected after 0, 1, 2, 3, 4, 8, 12 and 24 weeks of incubation. The final data consisted of the mean of three-independent measurements. Particle size was calculated by the manufacturer's software.

### Example 8.3

### High Performance Liquid Chromatography

The free-fractions of insulin glargin and hAmyPEG5k in combinations were assessed by a C18-reversed phase high performance liquid chromatography (C18-RP-HPLC) using a Thermo Scientific C18 column (ODS Hypersil, 250×4.6 mm, SN 0593286M lot 9418, Particle Size 5 um) with a flow rate of 4 mL/min, in a Jasco LC-2000 HPLC (Jasco Inc, USA). The samples were analyzed after 0, 3 and 6 months of incubation at 4° C. Approximately 20 uL of each sample was mixed with 80 uL of 50% H₂O, 50% CH₃CN, 0.1% TFA solution prior to injection in a 20 uL stainless steel injection tubing loop. The purification gradient was performed as follow: 5 min of isocratic flow with 50% CH₃CN in water containing 0.1% TFA, 15 min of linear gradient of CH₃CN in water containing 0.1% TFA, progressing from 50% to 80% followed by 10 min of isocratic flow with 100% CH₃CN in 0.1% TFA. The sample elution was monitored by following the absorbance at 220 nm. The amount of free-fraction was calculated by the area under the curve. Aggregates were estimated by subtracting the final peptide free-amount from the starting concentration.

What is claimed is:

1. A non-agglomerating bioconjugate of human amylin or amylin analogue, wherein said bioconjugate contains at least one acyl unit.

2. The non-agglomerating bioconjugate of human amylin of claim 1, wherein said one acyl unit is covalently bonded to the Lysine 1 residue of SEQ ID NO: 1.

3. The non-agglomerating bioconjugate of human amylin of claim 1, having the formula I
(R1-X)m-R2
wherein R1 represents an acyl moiety selected from the group consisting of acetyl, biotin, ubiquitin, glycans, fatty acid chains and natural or synthetic polymers, polyethylene glycol (PEG), and functional spacers with various average molar masses,
R2 represents SEQ ID NO: 1,
X represents NH, CO, or O,
m represents the number of units of the acyl groups (R1) conjugated to SEQ ID NO: 1 (R2) obtained from the acylation of SEQ ID NO: 1, including human amylin analogues bearing an amino acid substitution in one or more of positions 3, 4, 5 or 6 (SEQ ID NO: 2);

4. The non-agglomerating bioconjugate of human amylin of claim 3, wherein said acyl group is produced with at least one acylating agent is selected from the group consisting of PEG an acetyl group, palmitoyl, and myristoyl.

5. The non-agglomerating bioconjugate of human amylin of claim 4, wherein PEG is from 1 KDa to 40 Kda.

6. A composition comprising a non-agglomerating acylated bioconjugate of human amylin or amylin analogue.

7. The composition of claim 6, further comprising insulin, an insulin analogue, and/or a GLP-1 receptor agonist analogue; and a pharmaceutically acceptable carrier.

8. The composition of claim 7, wherein the insulin or insulin analogue is selected from the group consisting of NPH, regular insulin, Glargine, Detemir, Degludec, Aspart, Lispro, and Glulisine.

9. The composition of claim 7, wherein the GLP-1 receptor agonist analogue is selected from the group consisting of semaglutide, exenatide, lixisenatide, liraglutide, albiglutide, and dulaglutide.

10. The composition of claim 6, wherein said composition is used in the treatment of pancreatitis, hypercalcemia, pain, osteoporosis, chronic inflammatory diseases, coeliac disease, psoriasis, diseases or conditions caused or favored by amyloid deposition or accumulation that leads to dysfunction or failure of systemic organs, and disorders and vascular diseases resulting from increase in blood pressure.

11. The composition of claim 10, wherein said diseases or problems caused or favored by amyloid deposition or accumulation that leads to dysfunction or failure of systemic organs are selected from the group consisting of hyperglycemia, diabetes, low tolerance to glucose or deficient glucose metabolism, obesity, metabolic syndrome, central nervous system-associated or peripherally-associated feeding disorders and Alzheimer's disease.

12. The composition of claim 10, wherein said problems and vascular diseases resulting from increase in blood pressure are selected from the group consisting of atherosclerosis, myocardial infarction, stroke, coronary heart disease, hypertension, and cardiac diseases.

13. The composition of claim 6, further comprising an anti-inflammatory.

14. A method for preparing a composition, comprising mixing the non-agglomerating bioconjugate of human amylin of claim 1, insulin, and a pharmaceutically acceptable carrier.

15. A medicament comprising a therapeutically effective amount of the non-agglomerating bioconjugate of human amylin of claim 2.

16. A method for treating a disease or condition caused by a lack of amylin or amyloid deposition or accumulation, comprising administering to a patient in need thereof the composition of claim 7.

17. The method of claim 16, wherein said disease is diabetes and/or obesity.

18. A method for stabilizing an amylin-mimetic compound, comprising binding an acylating agent at the alpha and/or epsilon amine moieties of the lysine 1 residue of the amylin polypeptide chain.
